(19) United States
(12) Patent Application Publication
(10) Pub. No.: US 2006/0040825 A1
(43) Pub. Date: Feb. 23, 2006

(54) Method of improving the growth of cotton

(75) Inventors: Michael Kenty, Collierville, TN (US); James Thomas, Cordova, TN (US); Greg Volgas, Bartlett, TN (US)

Correspondence Address: CONNOLLY BOVE LODGE & HUTZ, LLP, P O BOX 2207, WILMINGTON, DE, 19899, US

(73) Assignee: HELENA HOLDING COMPANY, Wilmington, DE (US)

(21) Appl. No.: 11/210,185

(22) Filed: Aug. 23, 2005

Related U.S. Application Data
(60) Provisional application No. 60/603,779, filed on Aug. 23, 2004.

(51) Int. Cl. A01H 3/00; A01N 25/00

(52) U.S. Cl. 504/116.1; 47/58.1 R

(57) ABSTRACT

A method of determining the most efficient timing for application of nitrogen to growing cotton plants, wherein the method has the following steps: a) Collecting cotton tissue samples from the cotton field, b) Extracting sap from the cotton tissue samples within 24 hours of sample collection, and c) Analyzing the sap within a short time of extraction, by measuring the nitrate nitrogen levels of cotton leaf sap using an ion specific electrode measuring device.

### BACKGROUND OF THE INVENTION

The ability to closely monitor the level of nutrients in plant tissue is critical to crop production in many crops, including cotton. Growers can use the analyses of plant tissue to prescribe foliar application of nutrients. In some cases, the labs that analyze the plant tissue will supply growers with foliar fertilization recommendations tailored for the fields from which the samples were pulled. Such recommendations are based on a level of nutrients that have historically provided the specific crop with the highest yields.

A major drawback of this approach is the length of time between tissue sampling and the reporting of lab results. Sampling and analysis on the same day could greatly improve the fertilizer recommendations provided for growers.

Universities and laboratories have already produced charts showing the optimum level of nutrients in cotton at each stage of cotton growth. The problem with these charts is that the data they represent is taken from crop nutrient analyses that may be out of date when the grower received the information.

Ion specific electrodes can be used by growers to analyze their own tissue samples for nutrient levels. Cardy meters (manufactured by Horiba, Ltd. in Kyoto, Japan) are one such meter that can be hand-carried into fields and used on the spot to measure cotton tissue samples. The Horiba-Cardy Nitrate Meter consists of a miniature ion-selective and reference electrode mounted in a replaceable plastic insert, and a two-digit LCD display. The instrument weighs approximately 40 grams and is 3.7 inches long, 2.2 inches wide, and 0.35 inches thick. The Manufacturer claims the following resolutions for nitrate-N determinations: 1 ppm for samples in the range of 0-99 ppm, 10 ppm for 100-990 ppm, and 100 ppm for 1000 to 9900 ppm. NitrateN standards of 450 and 20 ppm were used to calibrate the meter. Both the high and low standards were checked every six samples to correct for fluctuations in electrode sensitivity.

Several investigators have investigated the use of Cardy meters in the production of cotton. Burmester and Mullins (Evaluation of a compact ion meter for in-field measurement of potassium in cotton petioles. Proceedings of the Beltwide Cotton Conference. 1994. National Cotton Council, Memphis, Tenn. Page 1574); Hodges and Baker (Correlation of plant sap extracts of nitrate-N and K with dried petiole extracts. Proceedings of the Beltwide Cotton Conference. 1993. National Cotton Council, Memphis, Tenn. pages 1335-1337.) and Smith, et. al. (Comparison of the two methods for the analysis of petiole nitrate nitrogen concentration in irrigated cotton. Publication AZ1006. College of Agriculture, university of Arizona, Tuscon, Ariz.) These references are incorporated herein.

In 1993, Hodges and Baker studied the correlation between Cardy meter testing and lab analyses (Correlation of plant sap extracts of nitrate-N and K with dried petiole extracts. Proceedings of the Beltwide Cotton Conference. 1993. National Cotton Council, Memphis, Tenn. pages 1335-1337.) They determined that there was no siginificant difference between the analyses of the lab and that of the Cardy meter. They found that as cotton plants aged, it became harder to extract sufficient sap for a good analyses.

In 1997, Cardy meters were used in vegetable production to determine threshold levels of nitrate nitrogen and potassium in a variety of vegetable crops (Maynard and Hochmuth, 1997, Knott's Handbook for vegetable growers. 4^{th} Edition. John Wiley and Sons. New York, 1997). These references are also incorporated herein. Prior work on “trigger points” or threshold values for nutrients has concentrated on soil application of conventional fertilizers. These values are not necessarily accurate for foliar application or for methylene urea controlled release fertilizers.

Development work on this method has been conducted by Helena and its cooperators, over the last three years. However, this development work did not rely solely on instantaneous leaf tissue analyses. All field measurements of nitrogen and potassium were correlated with lab analysis.

### BRIEF SUMMARY OF THE INVENTION

An object of the invention is to develop a process that can instantly analyze the cotton leaves and determine the amount of nutrients that need to be applied to the cotton plant to optimize the growth of the cotton plant.

Another object of the invention is to develop a portable system that can be easily used on a cotton field and take readings of the levels of nitrate nitrogen and potassium at different locations of the field to determine the amount of foliar fertilizer that needs to be applied to the cotton plants at that location of the field.

A further object of the invention is A method of determining the most efficient timing for application of nitrogen and/or potassium to growing cotton plants, wherein the method comprises:
a. Collecting cotton tissue samples from the cotton field,
b. Extracting sap from the cotton tissue samples within 24 hours of sample collection, and
c. Analyzing the sap within 24 hours of extraction, by measuring the nitrate nitrogen levels of cotton leaf sap using an ion specific electrode measuring device.

### DESCRIPTION OF THE INVENTION

Typically, the cotton leaves are pulled from the plants in the field. The petioles of these leaves are normally the tissue used for taking nutrient analyses. Typically, the petioles are simply isolated form the rest of the leaf tissue, and then pressed using a common garlic press, or similar apparatus, to extract the sap. This sap is then tested using the Cardy meter to determine the nutrient level of the petiole. Note that all of this can be performed on site by growers, thus providing real-time nutrient data.

The analyzing is within 24 hours after extracting the sap from the samples, preferably within 5 hours and even more preferably within 2 hours and most preferably within one hour after extracting the sap from the samples. The analyzing can be performed on site by growers, thus providing real-time nutrient data.

Using a combination of cotton nutritional studies conducted at several locations, we have determined a way for growers to know immediately when they need to make nutrient applications. Growers collect leaf samples from various areas within a field of growing cotton. These leaf samples are then immediately analyzed for nitrate nitrogen and potassium. For each stage of cotton growth, “trigger-points” have been determined to help growers decide when to fertilize. The “trigger-points” are actually levels of nutrients found in plants using a Cardy meter, that tell a grower whether or not his plants need a foliar application of nutrients. At any point below the “trigger-point”, growers will apply nitrogen and/or potassium fertilizers. Using this method, growers can maximize the yield potential of their crop.

The nitrogen fertilizers useful in this method are listed in the 2004 Official Publication of AAPFCO (Association of American Plant Food Control Officials). Particularly useful for this method, are nitrogen fertilizers derived from urea-formaldehyde reactions. Urea-formaldehyde reaction products (also referred to as methylene urea products) are common control release fertilizers in the agricultural and horticultural fields. These products release urea nitrogen slowly, thereby allowing growers to obtain more efficient fertilization of their crops. Since the “trigger-points” were developed using this controlled release fertilizer as the foliar fertilizer, the specific “trigger-points” are preferably applicable when that type fertilizer is used.

The potassium fertilizers useful in this method are also listed in the 2004 Official Publication of AAPFCO. They include, but are not limited to, potassium chloride, potassium nitrate, potassium thiosulfate, potassium hydroxide and its salts, and potassium sulfate.

The following is a list of the trigger points

Critical Cardy Meter N and K Levels* for Cotton

Cardy Meter
Readings
in PPM

| Week of Bloom | NO₃—N | K |
|---|---|---|
| −1 | 1450 | 4380 |
| 0 | 1450 | 4380 |
| 1 | 1450 | 4380 |
| 2 | 1310 | 4100 |
| 3 | 1090 | 3810 |
| 4 | 820 | 3510 |
| 5 | 430 | 3200 |
| 6 | 430 | 3010 |
| 7 | 280 | 2760 |
| 8 | 210 | 2560 |

All the references described above are incorporated by reference in its entirety for all purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

We claim:

1. A method of determining the most efficient timing for application of nitrogen to growing cotton plants, wherein the method comprises:
a. collecting cotton tissue samples from the cotton field,
b. extracting sap from the cotton tissue samples within 24 hours of sample collection, and
c. analyzing the sap within 24 hours of extraction, by measuring the nitrate nitrogen levels of cotton leaf sap using an ion specific electrode measuring device.

2. The method as claimed in claim 1, wherein said extracting of sap occurs on said field.

3. The method as claimed in claim 2, wherein said analyzing said sap occurs on said field.

4. The method as claimed in claim 3, wherein said analyzing is within 5 hours after sampling.

5. The method as claimed in claim 3, wherein said analyzing is within 2 hours after sampling.

6. A method of increasing the growth of cotton plants which comprises determining the amount of nitrogen to be applied to the plant from the method as claimed in claim 1, and applying nitrogen at pre-set trigger points, wherein said nitrogen is derived at least in part from urea-formaldehyde solutions.

7. A method of determining the most efficient timing for application of potassium to growing cotton plants, wherein the method comprises:
a. measuring the potassium levels of cotton leaf sap using an ion specific electrode measuring device, and
b. applying a potassium-containing fertilizer at pre-set trigger points.

8. The method as described in claim 7 with further comprises:
a. collecting cotton tissue samples from the field,
b. extracting the sap from the plant tissue within 24 hours of sample collection, and
c. analyzing the sap is analyzed by measuring the potassium levels of cotton leaf sap using an ion specific electrode measuring device within 24 hours of extracting, and
d. applying potassium fertilizer at pre-set trigger points.

9. A method of determining the most efficient timing for application of both potassium and nitrogen to growing plants, wherein the method comprises:
a. measuring the potassium and nitrogen levels of cotton leaf sap using an ion specific measuring device, and
b. applying a combination of potassium- and nitrogen-containing fertilizer at pre-set trigger points, wherein said nitrogen fertilizer is derived at least in part from urea-formaldehyde solutions.

10. A method as described in claim 9 which further comprises:
a. collecting cotton tissue samples from the field,
b. within 24 hours collecting, extracting sap form the plant tissue, and
c. within 24 hours of extracting, analyzing the sap by measuring the nitrate nitrogen and potassium levels of cotton leaf sap using an ion specific electrode measuring device, and
d. applying potassium and nitrogen at pre-set trigger points, wherein said nitrogen is derived at least in part from urea-formaldehyde solutions.

11. The method as described in claim 6 in which the trigger points are taken from the chart below:
Critical Cardy Meter N and K Levels* for Cotton

| | Cardy Meter |
|---|---|
| | Readings in PPM |
| Week of Bloom | NO₃—N |
| −1 | 1450 |
| 0 | 1450 |
| 1 | 1450 |
| 2 | 1310 |
| 3 | 1090 |
| 4 | 820 |
| 5 | 430 |
| 6 | 430 |
| 7 | 280 |
| 8 | 210 |

12. The method as described in claim 7 in which the trigger points are taken from the chart below:
Critical Cardy Meter N and K Levels* for Cotton

| | Cardy Meter Readings |
|---|---|
| | in PPM |
| Week of Bloom | K |
| −1 | 4380 |
| 0 | 4380 |
| 1 | 4380 |
| 2 | 4100 |
| 3 | 3810 |
| 4 | 3510 |
| 5 | 3200 |
| 6 | 3010 |
| 7 | 2760 |
| 8 | 2560 |

13. The method as described in claim 9 in which the trigger points are taken from the chart below:
Critical Cardy Meter N and K Levels* for Cotton
Cardy Meter
Readings
in PPM

| Week of Bloom | NO₃—N | K |
|---|---|---|
| −1 | 1450 | 4380 |
| 0 | 1450 | 4380 |
| 1 | 1450 | 4380 |
| 2 | 1310 | 4100 |
| 3 | 1090 | 3810 |
| 4 | 820 | 3510 |
| 5 | 430 | 3200 |
| 6 | 430 | 3010 |
| 7 | 280 | 2760 |
| 8 | 210 | 2560 |

14. The method as described in claim 6 in which the urea-formaldehyde solution consists essentially of urea and urea-formaldehyde polymers and water.

15. The method as described in claim 6 in which the urea-formaldehyde solution consists essentially of urea and urea-formaldehyde polymers, potassium chloride and water.

16. The method as described in claim 15 wherein the urea-formaldehyde polymer is present in an amount of at least 10%.

17. The method as described in claim 15 wherein the urea-formaldehyde polymer is present in an amount of at least 5%.

18. The method as described in claim 6 in which the urea-formaldehyde solution comprises urea and urea-formaldehyde polymers, potassium carbonate and water.

19. The method as described in claim 9 in which the urea-formaldehyde solution comprises urea, urea-formaldehyde polymers, and potassium chloride.

20. The method as described in claim 9 in which the urea-formaldehyde solution comprises urea, urea-formaldehyde polymers, and potassium carbonate.

21. The method as described in claim 9 in which the urea-formaldehyde solution comprises urea, urea-formaldehyde polymers, and an agronomically effective potassium fertilizer.

22. The method as described in claim 1 which further comprises the use of remote sensing technology for agronomic diagnostics.

23. The method as described in claim 6 which further comprises the use of remote sensing technology for agronomic diagnostics.

24. The method as described in claim 8 which further comprises the use of remote sensing technology for agronomic diagnostics.

25. The method as described in claim 9 which further comprises the use of remote sensing technology for agronomic diagnostics.
